(19) United States
(12) Patent Application Publication
(10) Pub. No.: US 2012/0061274 A1
(43) Pub. Date: Mar. 15, 2012

(54) Hardpack Needle Package Laser Heat Seal

(75) Inventors: Santo Tumminello, Deland, FL (US); Jonathan G. Parker, Debary, FL (US)

(73) Assignee: TYCO HEALTHCARE GROUP LP, Mansfield, MA (US)

(21) Appl. No.: 12/881,270

(22) Filed: Sep. 14, 2010

(51) Int. Cl. A61B 17/06

(52) U.S. Cl. 206/438; 53/477

(57) ABSTRACT

Hardpack devices including a cartridge and a cap are useful for protecting sterile medical instruments. The cartridge includes a body having an open end and a closed end, and defines an internal cavity. The cap is adapted for placement over the open end of the body for enclosing the internal cavity of the cartridge. A laser heat seal mark is scored at the interface between the cartridge and the cap to seal the device.

### TECHNICAL FIELD

The present disclosure relates to packages for providing a sterile medical instrument in a tamper-evident container. En particular, the present disclosure relates to a needle hardpack including a laser heat seal mark, and methods of forming and using the same.

### BACKGROUND

Medical instruments, particularly those including a sharp surface such as needles and syringes, may be packaged in a hard plastic shell known as a rigid pack or a hardpack. The hardpack may include a container, sometimes referred to as a cartridge, and a cap or cover removably attached thereto, for closing the hardpack. The hardpack may be sealed, thereby providing a sterile barrier around the medical instrument.

In some cases, the seal may be produced by contact induction between the cap and the cartridge of the hardpack. The seal holds the cap and cartridge together and provides an indication once the cap has been removed by the misalignment of the mark produced by the contact induction process on the cap and cartridge. The seal may be formed by contacting a solder or resistance heater tip against the hardpack and melting the plastic of the cap and cartridge together. Plastic deformation occurs at the site of contact of the heater tip with the hardpack, thereby producing a mark. Removal of the heater tip from the hardpack may also overspill the plastic, thereby creating an increased outer diameter at the site of contact. This method may also result in the formation of plastic threads as the heater tip is pulled away from the surface of the hardpack.

To open a hardpack with a contact induction seal, a two step process of cracking the seal by applying a force on the opposite side of the seal, and then twisting the cap is required. Without applying the cracking force first, the twist off force can be 20 inch-ounces (in-oz) or more, which can be extremely difficult to apply by hand.

It would be advantageous to provide a seal in which the torque characteristics can be controlled to reduce the torque required to open the hardpack, thus simplifying the opening process. It would also be advantageous to provide a seal that leaves a “clean” mark without plastic displacement and build up.

### SUMMARY

The present disclosure provides hard plastic shells, referred to herein, in embodiments, as a rigid pack or a hardpack, suitable for holding a medical device or medicament. In embodiments, a hardpack of the present disclosure includes a cartridge including a body having an open end and a closed end, the body defining an internal cavity; a cap configured for placement upon the open end of the body; and at least one laser heat seal mark scored about an interface between the cartridge and the cap.

Methods for producing a hardpack of the present disclosure are also provided. In embodiments, a method of the present disclosure for forming a laser sealed hardpack may include assembling a cartridge and a cap; and applying laser energy at the cartridge and cap interface to form a laser heat seal mark.

### BRIEF DESCRIPTION OF THE DRAWINGS

Various embodiments of the hardpack devices of the present disclosure are described herein with reference to the drawings, in which:

FIG. 1A is a schematic illustration of a hardpack in accordance with an embodiment of the present disclosure;

FIG. 1B is a schematic illustration of the hardpack of FIG. 1A with parts separated;

FIG. 2 is a schematic illustration of a hardpack including multiple laser heat seal marks in accordance with an embodiment of the present disclosure; and

FIG. 3 is a schematic illustrate of a hardpack including a laser heat seal mark in accordance with another embodiment of the present disclosure.

### DETAILED DESCRIPTION

The hardpack devices of the present disclosure provide a sterile environment for medical instruments while providing a tamper-evident seal and control over the torque values required to open the devices. A hardpack includes a cartridge including a body defining an inner cavity for disposal of a medical instrument therein, and a cap for enclosing the inner cavity of the cartridge. The hardpack may house a variety of medical devices, such as, for example, injection and piercing devices like needles, syringes, needle and hub assemblies, needle and syringe assemblies, and the like. The hardpack may be sized and shaped to accommodate a desired medical device and may have any regular or irregular cross sectional shape including circular, elliptical, square, rectangular, or trapezoidal. There may, but need not, be one cross sectional shape or cross sectional area throughout the hardpack.

Referring now to the drawings, in which like reference numerals identify identical or substantially similar parts throughout the several views, embodiments of hardpack devices in accordance with the present disclosure are provided. While the description and drawings below depict hardpacks having an elongate sheath configuration, the methods of the present disclosure may be utilized to seal a hardpack of any shape or configuration.

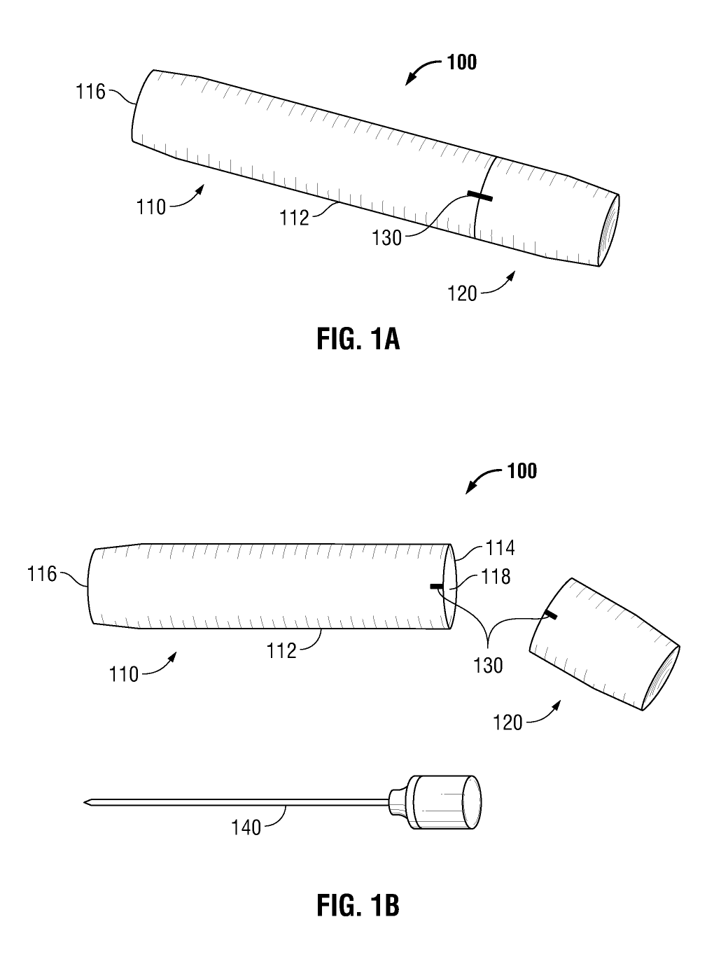

FIGS. 1A and 1B illustrate an embodiment of a hardpack, referred to generally by reference numeral 100. The hardpack 100 includes a cartridge 110 and a cap 120. The cartridge 110 includes an elongate sheath body 112 having an open end 114 and a closed end 116, and defines an internal cavity 118. The open end 114 of the elongate sheath body 112 may be a generally cylindrical opening having a predetermined diameter for the introduction of a medical instrument 140 therethrough. The internal cavity 118 serves as a receptacle or vessel for storing the medical instrument 140. Cap 120 is releasably connectable to the open end 114 of the elongate sheath body 112 for enclosing the internal cavity 118 of the cartridge 110. The inner diameter of the cap 120 may be sized and dimensioned to accommodate the outer diameter of the open end 114 of the elongate sheath body 112. In embodiments, the open end 114 of the elongate sheath body 112 and the cap 120 may be configured to include mating structures to secure the cap 120 on the cartridge 110, such as by snap fit or friction fit, for example. Alternatively, the body 112 and cap 120 may include mating structures such as threads forming a screw cap.

The cartridge 110 and the cap 120 may be fabricated from puncture resistant polymeric materials that are capable of absorbing laser energy as described herein. The polymeric materials may be crystalline or semi-crystalline materials. Alternatively, the polymeric materials may be amorphous materials. Laser treatment may, but need not, change the crystallinity of the materials. In one embodiment, the cartridge 110 and the cap 120 may be formed of the same material. In another embodiment, the cartridge 110 and the cap 120 are formed of different materials having substantially similar melt characteristics.

Suitable materials from which the cartridge and/or cap may be fabricated include, but are not limited to, polyolefins such as polyethylene (including ultra high molecular weight polyethylene) and polypropylene including atactic, isotactic, syndiotactic, and blends thereof; polyethylene glycols (PEGs); polyethylene oxides; copolymers of polyethylene and polypropylene; polyisobutylene and ethylene-alpha olefin copolymers; fluorinated polyolefins such as fluoroethylenes, fluoropropylenes, fluoroPEGs, and polytetrafluoroethylene; polyamides; polyamines; polyimines; polyesters such as polyethylene terephthalate, polyethylene naphthalate, polytrimethylene terephthalate, and polybutylene terephthalate; polyethers; polybutester; polytetramethylene ether glycol; 1,4-butanediol; polyurethanes; acrylic polymers; methacrylics; vinyl halide polymers and copolymers such as polyvinyl chloride; polyvinyl alcohols; polyvinyl ethers such as polyvinyl methyl ether; polyvinylidene halides such as polyvinylidene fluoride and polyvinylidene chloride; polychlorofluoroethylene; polyacrylonitrile; polyaryletherketones; polyvinyl ketones; polyvinyl aromatics such as polystyrene; polyvinyl esters such as polyvinyl acetate; copolymers of vinyl monomers with each other and olefins such as ethylene-methyl methacrylate copolymers; acrylonitrile-styrene copolymers; acrylonitrile butadiene styrene resins; ethylene-vinyl acetate copolymers; alkyd resins; polycarbonates; polyoxymethylenes; polyphosphazines; polyimides; epoxy resins; aramids; silicones; and copolymers and combinations thereof.

Returning to FIGS. 1A and 1B, hardpack 100 includes at least one laser heat seal mark 130 is scored about the interface between the cartridge 110 and the cap 120 such that the laser heat seal mark extends thereacross. The laser heat seal mark 130 is utilized to seal the cartridge 110 and the cap 120, as well as to provide indicia that the cartridge 110 and the cap 120 have not been opened, thus protecting against instrument contamination and providing a particulate-free and sterile environment for the medical instrument.

The laser heat seal mark 130 may be provided by any laser that is capable of delivering sufficient energy to the hardpack 100 to heat and melt the area around the cartridge and cap interface without penetrating the inner surface of the cap 120 and the inner surface of the cartridge 110. Thus, the laser heats only a select surface area of the hardpack while not substantially heating the remainder of the hardpack and/or medical device. In embodiments, the surface of the hardpack is heated to about the onset or peak melting temperature of the polymeric material utilized to form the cap 120 and cartridge 110 of the hardpack 100.

The laser may be focused on or near the surface of the hardpack at the cartridge and cap interface. In embodiments, the laser is focused to a depth of not more than about 1 millimeter below the surface of the hardpack, in some embodiments not more than about 0.5 millimeters below the surface of the hardpack, and in yet other embodiments about 0.1 millimeters below the surface of the hardpack. Alternatively, a larger portion of the thickness of the hardpack may be heated. In embodiments, less than about 25% of the thickness of the hardpack is heated, in other embodiments less than about 15% of the thickness of the hardpack is heated, and in yet other embodiments less than about 5% of the thickness of the hardpack is heated.

In embodiments, lasers suitable for use with the present disclosure include lasers which produce visible red light or infrared light. In embodiments, gas lasers may provide energy in the mid-to-far infrared range, e.g., from about 1 μm to about 30 μm, in embodiments from about 5 μm to about 15 μm, and in other embodiments about 10 μm. Lasers capable of infrared emission include, for example, helium, helium-neon, and carbon dioxide (CO₂) lasers. Additional lasers capable of infrared emission include diode lasers, infrared neodymium lasers, and solid state lasers, such as neodymium:YAG lasers. Suitable lasers are within the purview of those skilled in the art and include those that are commercially available.

In embodiments, the laser can have a nominal power output of from about 10 watts to about 100 watts, in embodiments from about 20 watts to about 50 watts. Larger or thicker hardpacks may require lasers having a greater power output than a laser for use with smaller or thinner hardpacks. The actual power, however, applied to the hardpack may be less. For example, the power of the laser energy applied to the hardpack may be from about 5 watts to about 30 watts, in embodiments from about 10 watts to about 15 watts.

The beam width of the laser may vary. In embodiments, the beam width may be from about 0.05 millimeters to about 5 millimeters, in embodiments from about 0.1 millimeters to about 1 millimeter, and in other embodiments about 0.5 millimeters. In some embodiments, a defocused laser beam may be employed to provide energy over a larger area of the hardpack.

The laser may be continuous or pulsed. In embodiments utilizing a pulsed laser beam, the pulse duration of the laser beam may be less than about 0.05 seconds per pulse, in embodiments less than about 0.01 seconds per pulse, and in other embodiments about 0.005 seconds per pulse.

The selection of a laser for use according to the present disclosure will be determined, at least in part, by factors such as the absorption spectrum of the polymeric materials utilized to form the cartridge and the cap, the onset and peak melting or glass transition temperature of the polymeric material, the emission wavelength of the laser, the focus depth of the laser, the power output of the laser, the laser beam width, the time for which the laser is applied (and whether the laser beam is pulsed or continuous), combinations thereof, and the like.

In embodiments, a hardpack may be positioned to receive laser energy by securing the hardpack in a jig or other holder for treatment with a laser. In embodiments, the hardpack is held in a well-defined position to enable precise aim of the laser beam on the selected portion of the device. Sufficient laser energy is supplied for a period of time at a power level sufficient to heat the select portion of the hardpack and cause the polymer of the cap to melt together with the polymer of the elongated sheath body of the cartridge.

The laser energy may be provided to the hardpack in any desired location or pattern using conventional control means. For example, galvanometers and other control means can be used in combination with mirrors to control the location of the laser beam. A galvanometer in combination with a movable mirror may be referred to as a scanner, and such scanners are within the purview of those skilled in the art. A pair of orthogonally-mounted scanners can be used to control the laser beam in two dimensions (x and y axes) and can be used to provide a variety of patterns of laser energy to the hardpack. Thus, scanners and similar systems, as well as beam splitters and other apparatus within the purview of those skilled in the art, can be used to control the laser beam or beams utilized in the present disclosure. In embodiments, computerized controls may be used to provide automated control of the laser system. Such control systems are often employed in laser systems used for cutting or etching materials such as plastics, and such conventional controls may be readily adapted for use in the present disclosure.

The laser energy can be scanned in any desired pattern or shape over the surface of the hardpack. Thus, the laser heat seal mark 130 may include any structure that is tactually or visually perceptible by a clinician and extends across the cartridge 110 and cap 120 interface. As illustrated in FIGS. 1A and 1B, the laser heat seal mark 130 may be scored to linearly extend across a portion of the surface of the cap 120 and the elongate sheath body 112 of the cartridge 110. It should be appreciated that the number, width, and size of the marks 130, as well as the spacing between the marks 130, may vary depending on the parameters of the laser and the torque desired to open the hardpack.

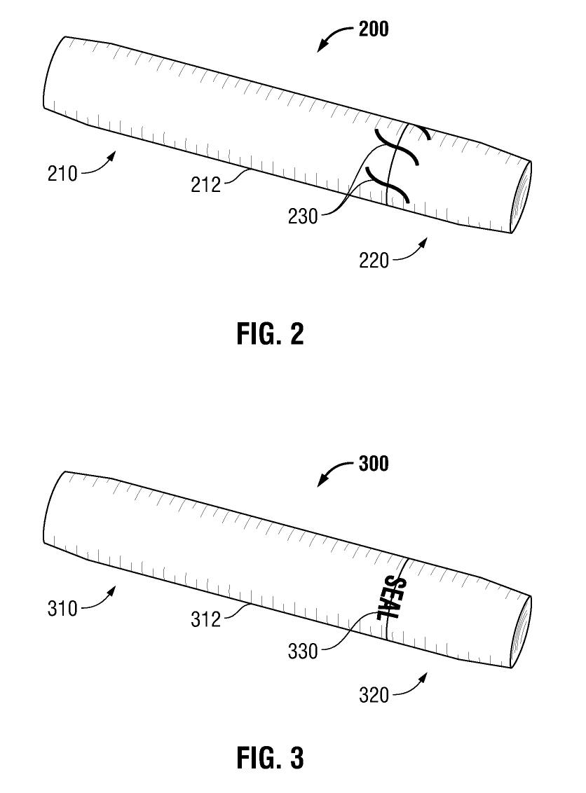

FIG. 2 illustrates an embodiment of a hardpack 200 which is similar to hardpack 100 seen in FIG. 1, but includes multiple laser heat seal marks 230 at the interface between the elongated sheath body 210 and the cap 220. As illustrated in FIG. 2, three non-linear marks 230 are shown. The marks may include a variety of other indicia, such as shapes, symbols, numerals, and/or text. As an illustrative example, FIG. 3 shows a hardpack 300 including a laser heat seal mark 330 including the text “SEAL.” Alternatively, the mark may be a continuous mark about the perimeter of the cartridge and cap interface.

The torque required to open a hardpack that has been sealed with a laser in accordance with the present disclosure may thus be adjusted depending upon the medical instrument stored within the hardpack and its intended use, which may correspond to the amount of force needed to open the hardpack. Overall, the amount of torque required to open a hardpack sealed in accordance with the present disclosure may be from about 3 in-oz to about 30 in-oz, in embodiments from about 6 in-oz to about 20 in-oz.

In embodiments, the polymers forming the hardpack may contain visualization agents to improve the visibility of the seal. In embodiments, the visualization agent may be along or adjacent to the laser heat seal mark. Visualization agents may be selected from a variety of non-toxic colored substances, such as dyes. Suitable dyes are within the purview of those skilled in the art and may include, for example, FD&C Blue #1, FD&C Blue #2, FD&C Blue #3, FD&C Blue #6, D&C Green #6, methylene blue, indocyanine green, other colored dyes, and combinations thereof. It is envisioned that additional visualization agents may be used such as fluorescent compounds (e.g., flurescein or eosin). In embodiments, the laser heat seal marks may be colored to more clearly visualize the marks on the surface of the device. In embodiments utilizing more than one mark, each mark may be a different color for visualization of the alignment and integrity of the seal. It is envisioned that the visualization agent may become apparent upon sterilization, for example by heating such as autoclaving, and thus the visualization agent may also provide an indication that the medical instrument disposed within the hardpack is sterile.

Methods of making the laser heat seal marks are within the purview of those skilled in the art and include, but are not limited to, the techniques given as examples in this disclosure.

The following Examples are being submitted to illustrate embodiments of the present disclosure. These Examples are intended to be illustrative only and are not intended to limit the scope of the present disclosure.

### EXAMPLES

The following non-limiting example shows how laser parameters may be changed to alter the desired torque required to open a sealed hardpack.

A CO₂ laser (model ML-G9310) commercially available from Keyence Corporation, Woodcliff Lake, N.J., was utilized to score a heat seal mark on a hardpack sold under the trademark MONOJECT™ 250E by Kendall Corp., a division of Covidien. It was desired to provide torque in a range of about 2 in-oz to about 7 in-oz.

Twenty-three trials were run at various laser settings as indicated in Table 1 below. Each trial included making one linear mark at the cartridge and cap interface of the hardpack at the setting shown in Table 1.

TABLE 1

| | | | | Block | | | | | | | | |
|---|---|---|---|---|---|---|---|---|---|---|---|---|
| | Character Size | | | Position | | | | | | | | |
| | (mm) | | | (mm) | | Character | Approach | Skip Cross | | | Power | Speed |
| Trial | Height | Width | Space | X | Y | Line Type | (mm) | (mm) | | | (%) | (mm/s) |
| 1 | 3 | 2 | 2.5 | −5.75 | −24 | thin | 1 | 0 | 0 | 0 | 100 | 50 |
| 2 | 3 | 2 | 2.5 | −5.75 | −24 | thin | 1 | 0 | 0 | 0 | 75 | 50 |
| 3 | 3 | 2 | 2.5 | −5.75 | −24 | thin | 1 | 0 | 0 | 0 | 50 | 50 |
| 4 | 3 | 2 | 2.5 | −7.75 | −24 | thin | 1 | 0 | 0 | 0 | 100 | 50 |
| 5 | 3 | 2 | 2.5 | −4.75 | −24 | thin | 1 | 0 | 0 | 0 | 100 | 50 |
| 6 | 3 | 2 | 2.5 | −4.75 | −24 | thin | 1 | 0 | 0 | 0 | 100 | 50 |
| 7 | 3 | 3 | 2.5 | 5 | −24 | thin | 1 | 0 | 0 | 0 | 100 | 50 |
| 8 | 3 | 3 | 2.5 | 5.25 | −24 | thin | 1 | 0 | 0 | 0 | 100 | 50 |
| 9 | 3 | 3 | 2.5 | 5.25 | −24 | thin | 1 | 0 | 0 | 0 | 75 | 50 |
| 10 | 3 | 3 | 2.5 | 5.25 | −24 | thin | 1 | 0 | 0 | 0 | 50 | 50 |
| 11 | 3 | 3 | 2.5 | 5.25 | −24 | thin | 1 | 0 | 0 | 0 | 60 | 50 |
| 12 | 3 | 3 | 2.5 | 5.25 | −24 | thin | 1 | 0 | 0 | 0 | 55 | 50 |
| 13 | 3 | 3 | 2.5 | 5.25 | −24 | thick | 1 | 0 | 0.4 | 3 | 75 | 300 |
| 14 | 3 | 3 | 2.5 | 5.25 | −24 | thick | 1 | 0 | 0.4 | 3 | 75 | 250 |
| 15 | 3 | 3 | 2.5 | 5.25 | −24 | thick | 1 | 0 | 0.5 | 3 | 75 | 300 |
| 16 | 3 | 3 | 2.5 | 5.25 | −24 | thick | 1 | 0 | 0.6 | 3 | 75 | 300 |
| 17 | 5 | 3 | 2.5 | 5.25 | −24 | thick | 1 | 0 | 0.6 | 3 | 75 | 300 |
| 18 | 6 | 3 | 2.5 | 5.25 | −24 | thick | 1 | 0 | 0.6 | 3 | 75 | 300 |
| 19 | 6 | 3 | 2.5 | 5.25 | −24 | thin | 1 | 0 | 0.6 | 3 | 75 | 50 |
| 20 | 6 | 3 | 2.5 | 5.25 | −24 | thin | 1 | 0 | 0.6 | 3 | 75 | 100 |
| 21 | 6 | 3 | 2.5 | 5.25 | −24 | thin | 1 | 0 | 0.6 | 3 | 75 | 75 |
| 22 | 6 | 3 | 2.5 | 5.25 | −24 | thin | 0.1 | 0 | 0.6 | 3 | 75 | 60 |
| 23 | 6 | 3 | 2.5 | 5.25 | −24 | thin | 0.1 | 0 | 0.6 | 3 | 75 | 65 |

Each trial was run with five samples and the torque required to open each sample is provided in Table 2 below. The torque was measured using an AccuForce torque-check (from Ametek Inc.) (operating at from 0-200 in-oz). The sealed hardpack assembly was placed with the cartridge or sheath side in a stationary chuck device which contained the torque sensor. Next, a spring loaded locking device was lowered and secured to the top of the cap portion of the hardpack assembly. The instrument was then zeroed out and a pushbutton was pressed. This started turning an electric motor which created the torque that was applied to the cap. The motor continued to rotate the cap for one revolution, and then turned off. The peak torque value was recorded by the test instrument.

TABLE 2

| | Torque (in-oz) for Samples |
|---|---|
| Trial | 1, 2, 3, 4, 5 |
| 1 | 11, 15, 4, 15, 11 |
| 2 | 21, 11, 2, 15, 23 |
| 3 | 2, 2, 2, 66*, 7, 20 |
| 4 | 10, 9, 10, 16, 11, 8 |
| 5 | 13, 12, 14, 5, 6 |
| 6 | 12, 3, 13, 10, 3 |
| 7** | 15, 14, 12, 16 |
| 8 | 18, 17, 14, 16, 14 |
| 9 | 7, 11, 13, 17,8 |
| 10 | 8, 2, 4, 8, 2 |
| 11 | 4, 12, 13, 11, 13 |
| 12 | 10, 13, 14, 7, 8 |
| 13 | 2, 6, 3, 4, 7 |
| 14 | 3, 3, 6, 4, 6 |
| 15 | 8, 3, 6, 4, 3 |
| 16 | 4, 4, 4, 6, 6 |
| 17 | 4, 6, 6, 4, 3 |
| 18 | 6, 5, 3, 3, 6 |
| 19 | 7, 11, 10, 8, 12 |
| 20 | 2, 3, 6, 2, 4 |
| 21 | 4, 5, 4, 3, 2 |
| 22 | 7, 10, 9, 9, 6 |
| 23 | 7, 4, 6, 5, 6 |

*discounted reading
**one reading missed

As illustrated in Tables 1 and 2 above, changes in the laser setting parameters altered the torque required to open the hardpack.

An additional 30 samples were then evaluated at the laser setting of trial 23 as shown in Table 3 below.

TABLE 3

| | Torque |
|---|---|
| Sample | (in-oz) |
| 1 | 4 |
| 2 | 10 |
| 3 | 9 |
| 4 | 5 |
| 5 | 7 |
| 6 | 7 |
| 7 | 7 |
| 8 | 4 |
| 9 | 9 |
| 10 | 4 |
| 11 | 4 |
| 12 | 6 |
| 13 | 5 |
| 14 | 6 |
| 15 | 7 |
| 16 | 7 |
| 17 | 4 |
| 18 | 12 |
| 19 | 5 |
| 20 | 8 |
| 21 | 4 |
| 22 | 11 |
| 23 | 9 |
| 24 | 11 |
| 25 | 10 |
| 26 | 5 |
| 27 | 3 |
| 28 | 7 |
| 29 | 8 |
| 30 | 6 |

As illustrated in Table 3 above, the laser setting for trial 23 yielded torque of about 3 in-oz to about 11 in-oz with a 6.8 in-oz average to open the heat seal.

### COMPARATIVE EXAMPLES

Commercially available vials, sold as 250E vials by Covidien, having a conventional hardpack seal, were tested in the same way as described above in the Examples. 72 Samples were tested, with the results set forth below in Table 4.

The data was collected to see if the 250E cap or cartridge dimensions, as defined by the mold cavities, would have any effect on the torque off force. A 3 factor orthogonal test matrix was prepared with 2×3×3 levels, with the first two level factors being sterilization, and the two three level factors being cartridge mold number and cap mold number. The torque units were in in-oz. As can be seen from the comparative data, the Comparative Examples required higher torque to open the heat seal. This supports the conclusion that factors of sterilization or mold number were not significant.

TABLE 4

| | C1 | C2 | C3 | C4 | C5 | C6 | C7 | C8 | C9 |
|---|---|---|---|---|---|---|---|---|---|
| | Sterilation | Cartridge | Cap | Torque | StdOrder | RunOrder | Blocks | PtType | StdDev |
| 1 | 0 | 111 | 29 | 16 | 1 | 1 | 1 | 1 | 3.3040 |
| 2 | 0 | 111 | 31 | 22 | 2 | 2 | 1 | 1 | 2.6300 |
| 3 | 0 | 111 | 32 | 29 | 3 | 3 | 1 | 1 | 7.3655 |
| 4 | 0 | 112 | 29 | 14 | 4 | 4 | 1 | 1 | 3.5590 |
| 6 | 0 | 112 | 31 | 19 | 5 | 5 | 1 | 1 | 2.7080 |
| 6 | 0 | 112 | 32 | 20 | 6 | 6 | 1 | 1 | 2.8723 |
| 7 | 0 | 113 | 29 | 15 | 7 | 7 | 1 | 1 | 4.7958 |
| 8 | 0 | 113 | 31 | 6 | 8 | 8 | 1 | 1 | 7.7244 |
| 9 | 0 | 113 | 32 | 23 | 9 | 9 | 1 | 1 | 4.8990 |
| 10 | 1 | 111 | 29 | 19 | 10 | 10 | 1 | 1 | 1.2910 |
| 11 | 1 | 111 | 31 | 37 | 11 | 11 | 1 | 1 | 11.5866 |
| 12 | 1 | 111 | 32 | 19 | 12 | 12 | 1 | 1 | 2.2174 |
| 13 | 1 | 112 | 29 | 25 | 13 | 13 | 1 | 1 | 4.5092 |
| 14 | 1 | 112 | 31 | 22 | 14 | 14 | 1 | 1 | 3.3665 |
| 15 | 1 | 112 | 32 | 11 | 15 | 15 | 1 | 1 | 6.6521 |
| 16 | 1 | 113 | 29 | 12 | 16 | 16 | 1 | 1 | 2.4495 |
| 17 | 1 | 113 | 31 | 19 | 17 | 17 | 1 | 1 | 2.5000 |
| 18 | 1 | 113 | 32 | 18 | 18 | 18 | 1 | 1 | |
| 19 | 0 | 111 | 29 | 20 | 19 | 19 | 1 | 1 | |
| 20 | 0 | 111 | 31 | 21 | 20 | 20 | 1 | 1 | |
| 21 | 0 | 111 | 32 | 20 | 21 | 21 | 1 | 1 | |
| 22 | 0 | 112 | 29 | 19 | 22 | 22 | 1 | 1 | |
| 23 | 0 | 112 | 31 | 24 | 23 | 23 | 1 | 1 | |
| 24 | 0 | 112 | 32 | 17 | 24 | 24 | 1 | 1 | |
| 25 | 0 | 113 | 29 | 14 | 25 | 25 | 1 | 1 | |
| 28 | 0 | 113 | 31 | 16 | 26 | 26 | 1 | 1 | |
| 27 | 0 | 113 | 32 | 13 | 27 | 27 | 1 | 1 | |
| 28 | 1 | 111 | 29 | 18 | 28 | 28 | 1 | 1 | |
| 29 | 1 | 111 | 31 | 22 | 29 | 29 | 1 | 1 | |
| 30 | 1 | 111 | 32 | 21 | 30 | 30 | 1 | 1 | |
| 31 | 1 | 112 | 29 | 24 | 31 | 31 | 1 | 1 | |
| 32 | 1 | 112 | 31 | 18 | 32 | 32 | 1 | 1 | |
| 33 | 1 | 112 | 32 | 14 | 33 | 33 | 1 | 1 | |
| 34 | 1 | 113 | 29 | 24 | 34 | 34 | 1 | 1 | |
| 35 | 1 | 113 | 31 | 16 | 35 | 35 | 1 | 1 | |
| 36 | 1 | 113 | 32 | 19 | 36 | 36 | 1 | 1 | |
| 37 | 0 | 111 | 29 | 21 | 37 | 37 | 1 | 1 | |
| 38 | 0 | 111 | 31 | 20 | 38 | 38 | 1 | 1 | |
| 39 | 0 | 111 | 32 | 21 | 39 | 39 | 1 | 1 | |
| 40 | 0 | 112 | 29 | 14 | 40 | 40 | 1 | 1 | |
| 41 | 0 | 112 | 31 | 18 | 41 | 41 | 1 | 1 | |
| 42 | 0 | 112 | 32 | 20 | 42 | 42 | 1 | 1 | |
| 43 | 0 | 113 | 29 | 21 | 43 | 43 | 1 | 1 | |
| 44 | 0 | 113 | 31 | 24 | 44 | 44 | 1 | 1 | |
| 45 | 0 | 113 | 32 | 23 | 45 | 45 | 1 | 1 | |
| 46 | 1 | 111 | 29 | 17 | 46 | 46 | 1 | 1 | |
| 47 | 1 | 111 | 31 | 19 | 47 | 47 | 1 | 1 | |
| 48 | 1 | 111 | 32 | 17 | 48 | 48 | 1 | 1 | |
| 49 | 1 | 112 | 29 | 15 | 49 | 49 | 1 | 1 | |
| 50 | 1 | 112 | 31 | 11 | 50 | 50 | 1 | 1 | |
| 51 | 1 | 112 | 32 | 19 | 51 | 51 | 1 | 1 | |
| 52 | 1 | 113 | 29 | 18 | 52 | 52 | 1 | 1 | |
| 53 | 1 | 113 | 31 | 14 | 53 | 53 | 1 | 1 | |
| 54 | 1 | 113 | 32 | 22 | 54 | 54 | 1 | 1 | |
| 55 | 0 | 111 | 29 | 24 | 55 | 55 | 1 | 1 | |
| 56 | 0 | 111 | 31 | 26 | 56 | 56 | 1 | 1 | |
| 57 | 0 | 111 | 32 | 11 | 57 | 57 | 1 | 1 | |
| 58 | 0 | 112 | 29 | 21 | 58 | 58 | 1 | 1 | |
| 59 | 0 | 112 | 31 | 19 | 59 | 59 | 1 | 1 | |
| 60 | 0 | 112 | 32 | 14 | 60 | 60 | 1 | 1 | |
| 61 | 0 | 113 | 29 | 24 | 61 | 61 | 1 | 1 | |
| 62 | 0 | 113 | 31 | 20 | 62 | 62 | 1 | 1 | |
| 63 | 0 | 113 | 32 | 17 | 63 | 63 | 1 | 1 | |
| 64 | 1 | 111 | 29 | 20 | 64 | 64 | 1 | 1 | |
| 65 | 1 | 111 | 31 | 9 | 65 | 65 | 1 | 1 | |
| 66 | 1 | 111 | 32 | 16 | 66 | 66 | 1 | 1 | |
| 67 | 1 | 112 | 29 | 22 | 67 | 67 | 1 | 1 | |
| 68 | 1 | 112 | 31 | 22 | 68 | 68 | 1 | 1 | |
| 69 | 1 | 112 | 32 | 16 | 69 | 69 | 1 | 1 | |
| 70 | 1 | 113 | 29 | 27 | 70 | 70 | 1 | 1 | |
| 71 | 1 | 113 | 31 | 19 | 71 | 71 | 1 | 1 | |
| 72 | 1 | 113 | 32 | 16 | 72 | 72 | 1 | 1 | |

It should be understood that various lasers may be used to make a laser heat seal mark or marks on a variety of different hardpacks to obtain a desired torque according to the present disclosure. For example, the laser described in the example above may be utilized with a hardpack of a different material and wall thickness to provide a custom heat seal mark with the desired torque characteristics.

While several embodiments of the disclosure have been described herein, it is not intended that the disclosure be limited thereto, as it is intended that the disclosure be as broad in scope as the art will allow and that the specification be read likewise. Therefore, the above description should not be construed as limiting, but merely as exemplifications of embodiments of the present disclosure. Various modifications and variations of the hardpack device, as well as the size, number, and type of laser heat seal mark(s), and methods of forming the laser heat seal mark(s), will be apparent to those skilled in the art from the foregoing detailed description. Such modifications and variations are intended to come within the scope and spirit of the claims appended hereto.

What is claimed is:

1. A hardpack comprising:
a cartridge including a body having an open end and a closed end, the body defining an internal cavity;
a cap configured for placement upon the open end of the body; and
at least one laser heat seal mark scored about an interface between the cartridge and the cap.

2. The hardpack according to claim 1, wherein the open end of the elongate sheath body and the cap include mating structures for joining the cartridge and the cap.

3. The hardpack according to claim 1, wherein the cartridge and the cap are fabricated from puncture resistant materials selected from the group consisting of polypropylene, polyethylene, polyethylene terephthalate, polycarbonates, acrylonitrile butadiene styrene, polyvinylidene chloride, copolymers and combinations thereof.

4. The hardpack according to claim 1, wherein the laser heat seal mark comprises a linear mark.

5. The hardpack according to claim 1, wherein the laser heat seal mark is selected from the group consisting of shapes, symbols, numerals, text, and combinations thereof.

6. The hardpack according to claim 1, further comprising a visualization agent along the laser heat seal mark.

7. A method of forming a laser sealed hardpack comprising:
assembling a cartridge and a cap; and
applying laser energy at the cartridge and cap interface to form a laser heat seal mark.

8. The method according to claim 7, wherein applying laser energy at the cartridge and cap interface comprises focusing the laser at a depth of not more than about 1 millimeter below the surface of the hardpack.

9. The method according to claim 7, wherein applying laser energy at the cartridge and cap interface comprises focusing the laser at a depth of not more than about 0.5 millimeters below the surface of the hardpack.

10. The method according to claim 7, wherein applying laser energy at the cartridge and cap interface comprises heating less than about 25% of the thickness of the hardpack.

11. The method according to claim 7, wherein applying laser energy at the cartridge and cap interface comprises heating less than about 15% of the thickness of the hardpack.

12. The method according to claim 7, wherein applying laser energy at the cartridge and cap interface comprises heating less than about 5% of the thickness of the hardpack.

13. The method according to claim 7, wherein applying laser energy comprises applying a laser which ouputs energy in the infrared range.

14. The method according to claim 13, wherein the laser which outputs energy in the infrared range comprises a CO₂ laser.

15. The method according to claim 13, wherein the laser which outputs energy in the infrared range comprises a neodymium:YAG laser.

16. The method according to claim 7, wherein the laser energy is applied at a power of from about 5 watts to about 100 watts.

17. The method according to claim 7, wherein the laser energy is applied at a power of about 20 watts to about 50 watts.

18. The method according to claim 7, wherein the laser energy is applied with a beam width of from about 0.05 millimeters to about 5 millimeters.

19. The method according to claim 7, wherein the laser energy is applied with a beam width of from about 0.1 millimeters to about 1 millimeters.
